(19) United States
(12) Patent Application Publication
(10) Pub. No.: US 2024/0226423 A1
(43) Pub. Date: Jul. 11, 2024

(54) USER-WEARABLE INFUSION PUMP SYSTEM

(71) Applicant: Tandem Diabetes Care, Inc., San Diego, CA (US)

(72) Inventors: Jacob Pearlman, San Diego, CA (US); Neel Shah, San Diego, CA (US); Neal Johnston, San Diego, CA (US); Michael Michaud, San Diego, CA (US); Adam Evard, San Diego, CA (US); Eduardo Ho, San Diego, CA (US); Tinh Tran, San Diego, CA (US); Dominic Chiotti, San Diego, CA (US); Katalina Aguilar, San Diego, CA (US); Byron Cohea, San Diego, CA (US); Nathan Dale, San Diego, CA (US); Virginia Lu, San Diego, CA (US)

(21) Appl. No.: 18/407,663

(22) Filed: Jan. 9, 2024

Related U.S. Application Data
(60) Provisional application No. 63/437,872, filed on Jan. 9, 2023.

(51) Int. Cl. A61M 5/142

(52) U.S. Cl. CPC A61M 5/14248; A61M 2205/502

(57) ABSTRACT

Embodiments of the present disclosure relate to an infusion pump system including a holder or carrier for a user-wearable infusion pump that can be worn on the patient's body. Embodiments depicted and described herein provide a more versatile, secure, and user-friendly way to hold and carry a user-wearable infusion pump.

### PRIORITY CLAIM

The present application claims the benefit of U.S. Provisional Patent Application No. 63/437,872, which is hereby incorporated herein by reference in its entirety.

### TECHNICAL FIELD

The present disclosure relates to medical pumps for delivering medicament to a patient and, more specifically, to a user-wearable pump.

### BACKGROUND

There are a wide variety of medical treatments that include the administration of a therapeutic fluid in precise, known amounts at predetermined intervals. Devices and methods exist that are directed to the delivery of such fluids, which may be liquids or gases, which are known in the art.

One category of such fluid delivery devices includes insulin injecting pumps developed for administering insulin to patients afflicted with type I, or in some cases, type II diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

One type of pump that has been developed is a patch pump, or micro pump. Patch pumps generally are small pumps that are carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site. Not unlike other types of pumps, but perhaps more typically, patch pumps can be at least in part disposable, meant to be worn for a period of time such as, e.g., a day or two, and then discarded and replaced by a new patch pump. Other patch pump designs contemplate a disposable component, such as a cartridge that contains medicament, and a reusable or durable component. In such configurations, the disposable and durable components may be joined together by the patient or caregiver in preparation for delivery of the medicament. Still other patch pumps may include a rechargeable durable component as well as a refillable and reusable medicament cartridge.

### SUMMARY

Embodiments of the present disclosure relate to an infusion pump system including a holder or carrier for a user-wearable infusion pump that can be worn on the patient's body. Embodiments depicted and described herein provide a more versatile, secure, and user-friendly way to hold and carry a user-wearable infusion pump.

In an embodiment, a user-wearable infusion pump system includes a user-wearable infusion pump configured to contain a medicament and a pump holder configured to releasably hold the user-wearable infusion pump. The user wearable infusion pump can include a switch disposed on a perimeter of the user-wearable infusion pump that automatically releasably locks the user-wearable infusion pump onto the pump holder when the user-wearable infusion pump is inserted onto the pump holder.

In an embodiment, a user-wearable infusion pump configured to contain a medicament can include a switch disposed on a perimeter of the user-wearable infusion pump. The switch can be configured to automatically releasably lock the user-wearable infusion pump onto a pump holder when the user-wearable infusion pump is inserted onto the pump holder.

In an embodiment, a pump holder configured to releasably hold a user-wearable infusion pump is configured to cause a switch on a user-wearable infusion pump to automatically releasably lock the user-wearable infusion pump onto the pump holder when the user-wearable infusion pump is inserted onto the pump holder.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict a patch pump system according to an embodiment of the disclosure.

FIGS. 2A-2B depict a patch pump system according to an embodiment of the disclosure.

FIGS. 3A-3D depict a cartridge for a patch pump system according to an embodiment of the disclosure.

FIGS. 4A-4B depict a cartridge for a patch pump system according to an embodiment of the disclosure.

FIG. 5 depicts a patch pump system according to an embodiment of the disclosure.

FIGS. 6A-6C depict a patch pump system according to an embodiment of the disclosure.

FIGS. 7A-7C depict a patch pump system according to an embodiment of the disclosure.

FIG. 8 depict a patch pump system according to an embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

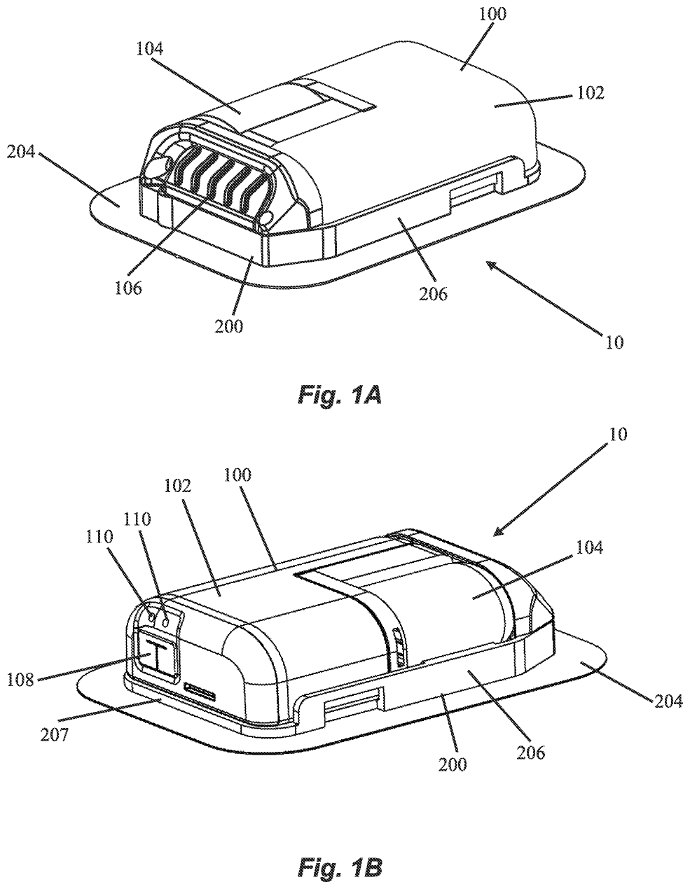

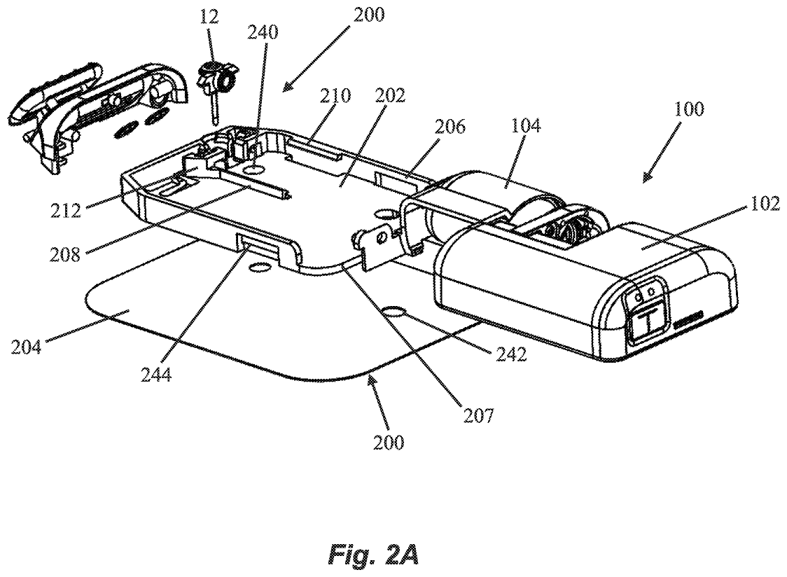

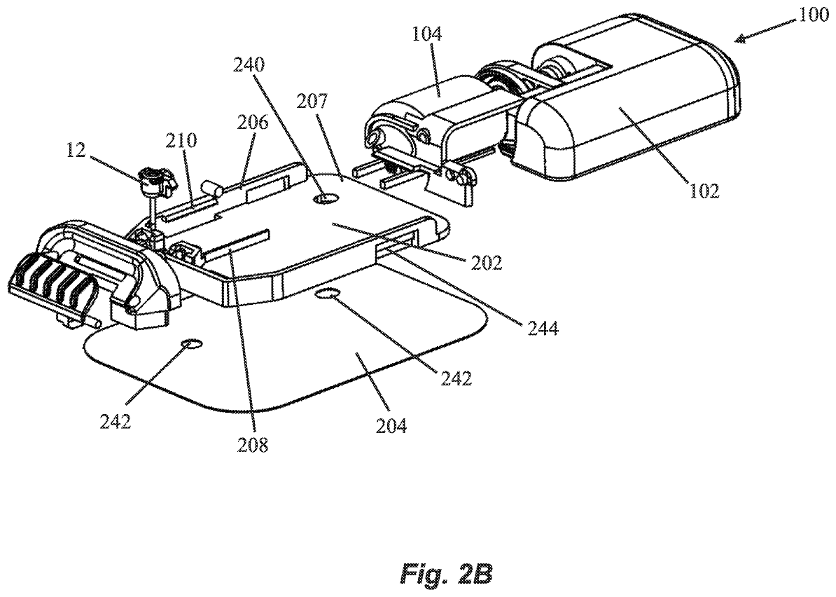

FIGS. 1A-1B and 2A-2B depict an ambulatory infusion pump system 10 according to an embodiment of the disclosure. System 10 can include an infusion pump 100 and a pump holder or tray 200 that can be used to releasably contain infusion pump 100 and that can be worn on a body of a user.

Pump 100 can include a drive unit 102 including a drive mechanism that mates with a recess in a medicament cartridge 104 to releasably hold the cartridge 104 on the drive unit 102. Further details regarding embodiments of a drive mechanism for delivering a medicament such as insulin from the medicament cartridge to a user and for attaching the cartridge to the drive unit can be found in U.S. Pat. Nos. 10,279,107 and 9,993,595, each of which is incorporated herein by reference in its entirety. In some embodiments, drive unit 102 is a durable component having a rechargeable battery and cartridge 104 is disposable and intended to be disposed of after the medicament in cartridge has been used. In other embodiments, cartridge can be refillable such that both components are durable. Pump 102 can further include a switch 106 configured to releasably lock the pump 102 onto the pump holder 200, as will be described in further detail below.

In embodiments, pump 100 can interface with a cannula 12 that extends through the pump holder 200 to deliver medicament to an infusion site on the user's body directly beneath the pump holder 200. In other embodiments, pump 100 can interface with infusion tubing to deliver medicament from the pump through the tubing to an infusion site displaced from the pump holder 200. In some embodiments, the pump 100 can be configured to interchangeably be used with a cannula directly beneath the pump holder 200 or an infusion site displaced from the holder 200 via infusion tubing. In some of those embodiments, drive unit 102 can be configured to attach to alternative cartridge types, with a first cartridge type including infusion tubing and a second cartridge type configured to deliver through a cannula extending directly beneath the pump 100. In various embodiments, pump 100 can be disconnected from a user's body and reattached using the same infusion site to enable the user to remove the pump for a limited period of time for certain activities, such as, e.g., charging the battery, taking a shower, playing sports, etc. without having to insert a cannula at a new infusion site.

In embodiments, pump includes a processor that controls the operations of the pump and may communicate in either one-way or two-way modes to, e.g., receive operational commands and/or other signals, including data, from a separate device and/or, e.g., to send signals, including data to a separate device. Pump can include on or more buttons configured to cause the processor to initiate one or more functions. In the depicted embodiment, pump 100 includes only a single button 108, although more than one button may be present on pump 100. Button 108 can be configured to, for example, initiate delivery of medicament (e.g., a “quick bolus”). Any single button such as button 108 can be utilized to execute a plurality of functions or operations. For example, a single press of button may initiate one function, holding the button down for a predetermined period of time may initiate another function, etc. Because the depicted pump 100 optionally does not itself include a display or user interface, information and feedback regarding medicament delivery or dosing or other functions initiated with button 108 can be communicated to and displayed on a remote control device or other device having a display and/or other type of user interface.

In embodiments, pump 100 includes a light source, such as a light emitting diode (LED) 110. Light source 110 can be configured to provide user feedback regarding user input and/or the performance of a desired function. For example, in one embodiment, light source 110 can illuminate or blink one or more times to indicate that the one or more buttons 108 have been activated and/or that a desired function has been initiated. In one embodiment, pump 100 can additionally and/or alternatively vibrate and/or provide audible notifications to indicate that the one or more buttons 108 have been activated and/or that a desired function has been initiated or, e.g., to provide user feedback regarding user input and/or the performance of the desired function. Illumination of light source 110 and/or vibrations and/or audible notifications may be executed in any number of patterns, frequencies, durations, sequences, combinations, colors, brightness levels, etc. to indicate particular information, such as particular input received and/or particular functions or operations enabled and/or initiated, to the pump user or caregiver. In the depicted embodiment, the pump 100 includes two indicator lights 110.

Pump holder 200 can include a tray body 202 having a shape generally matching the shape of pump 100 and an adhesive patch 204 configured to releasably retain the pump holder 200 and pump 100 on the user's body. One or more perimeter walls 206 can extend around at least a portion of the perimeter of the tray body 202 to aid in retaining and guiding the pump 100 onto the holder 200. In the depicted embodiment, tray body 202 can include one end 207 that does not include a perimeter wall to aid in sliding pump 102 onto tray body 202. Pump holder 200 can include various other features that aid in retaining and/or guiding the pump onto holder 200 that will be describe in more detail below, including a guide rail 208, retention projection 210 and a locking ramp 212. Pump holder 200 and patch 204 can include aligned apertures 240, 242 and retention walls 206 can include slots 244 that aid in temporarily retaining pump holder 200 in and releasing pump holder 200 from an insertion or applicator device configured to apply the pump holder 200 onto the user's skin. Further details regarding an applicator or insertion device can be found in U.S. Provisional Application No. 63/437,881 entitled Applicator Device for User-Wearable Infusion Pump, which is incorporated herein by reference in its entirety.

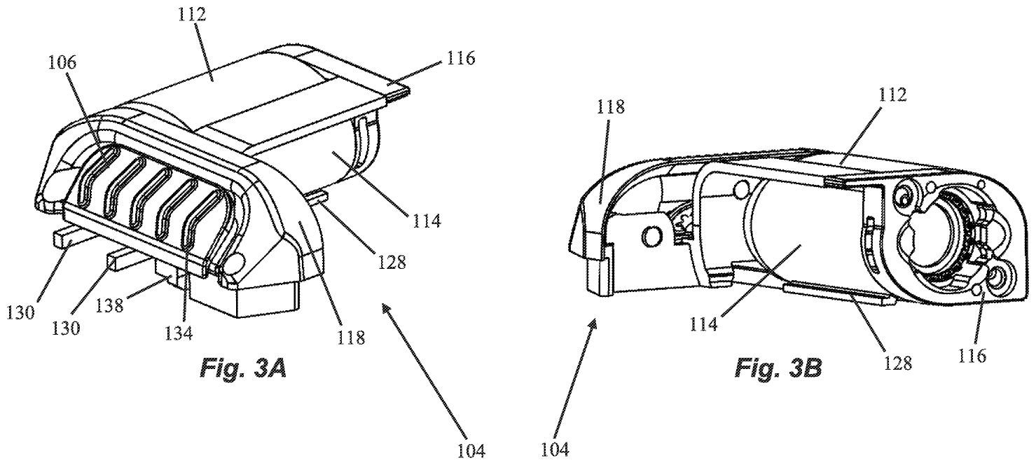

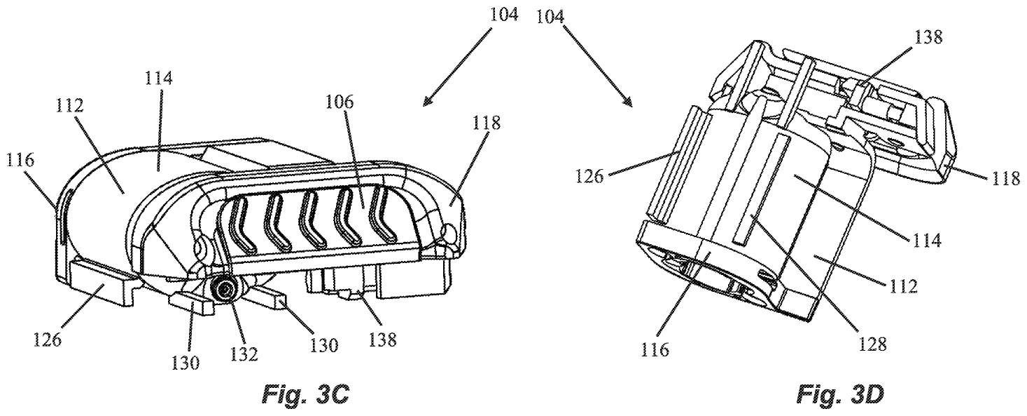

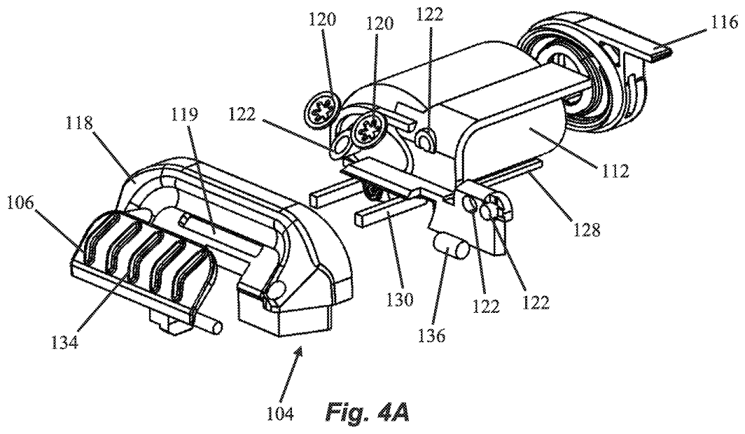

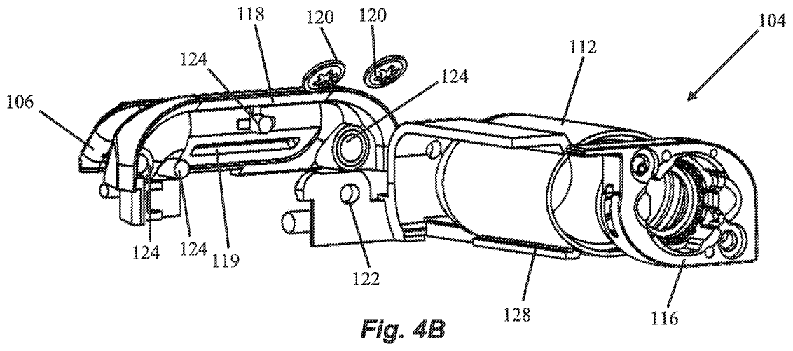

Further details regarding the configuration of cartridge 104 for infusion pump according to an embodiment are depicted in FIGS. 3A-3D and 4A-4B. Cartridge 104 can include a cartridge body 112 including a reservoir 114 for containing a medicament, an end cap 116 that interfaces with drive unit 102 and a faceplate 118 including switch 106. In this embodiment, the switch 106 can connect to the faceplate 118 with a pair of push nuts 120 that attach to corresponding projections on switch extended through slot 119 such that switch 106 can slide laterally with respect to faceplate 118 along slot 119. Faceplate and cartridge body 112 can include corresponding attachment features 122, 124 comprising a series of projections and corresponding recesses. In embodiments, the faceplate 118 can further be ultrasonically welded onto the cartridge body 112. End plate 116 can be press fit onto cartridge body 112.

Cartridge 104 further includes a number of features that aid in placement and retention of pump 100 on pump holder 200. A wall guide rail 126 can be disposed on an exterior surface of cartridge body 112. Wall guide rail 126 can be configured to slide under and be seated beneath the retention projection 210 of pump holder 200 to aid in guiding and retaining the pump 102 on the holder 200. A lower guide rail 128 can also be disposed on a bottom of cartridge body 112. Lower guide rail 128 can slide along guide rail 208 of holder 200 to properly align pump 100 on holder 200. A pair of sharps safety features 130 can extend outwardly from cartridge 104 positioned around a pocket 132 to protect a user from a needle that will extend from pocket 132 to interface the medicament reservoir 114 of cartridge 104 with the cannula 12 disposed in the holder.

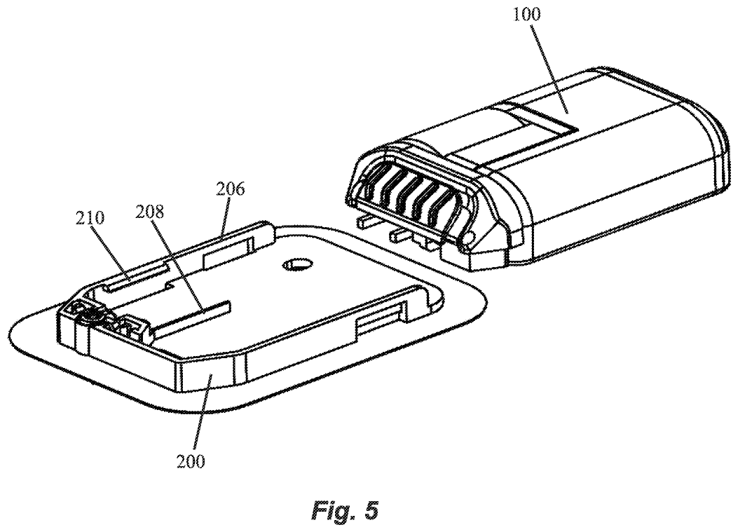

Referring to FIG. 5, pump 102 is designed to be inserted onto holder 200 by positioning pump adjacent the open end 207 and laterally sliding pump 100 along tray body 202. The interaction of lower guide rail 128 of pump sliding along guide rail 208 of holder 200 and wall guide rail 126 of pump along perimeter wall 126 and beneath retention projection 210 of holder ensures proper alignment of the pump 100 onto the holder 200.

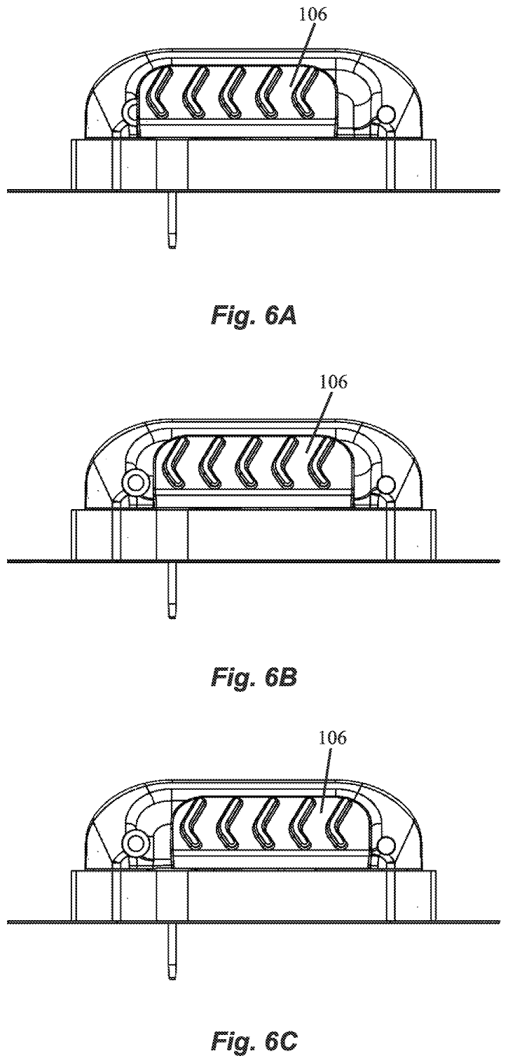

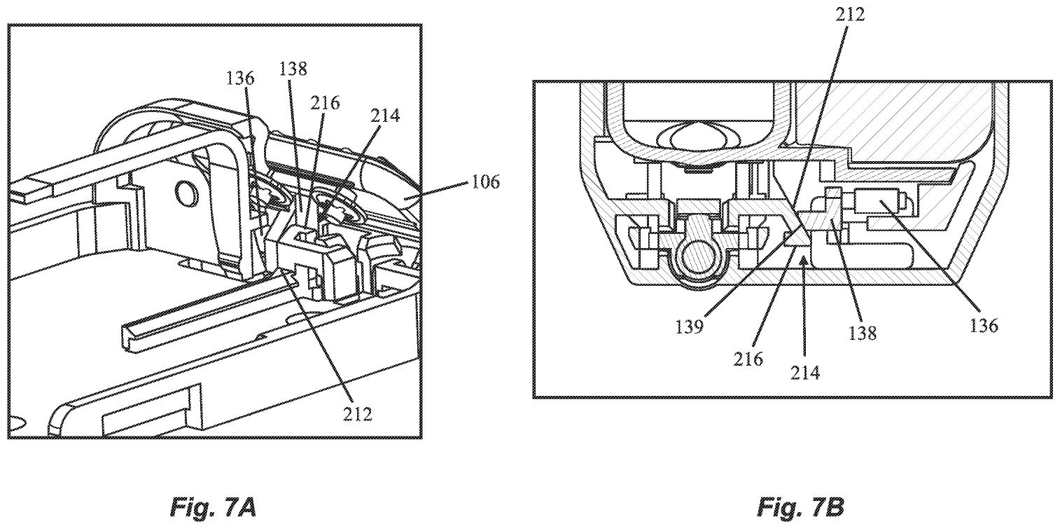

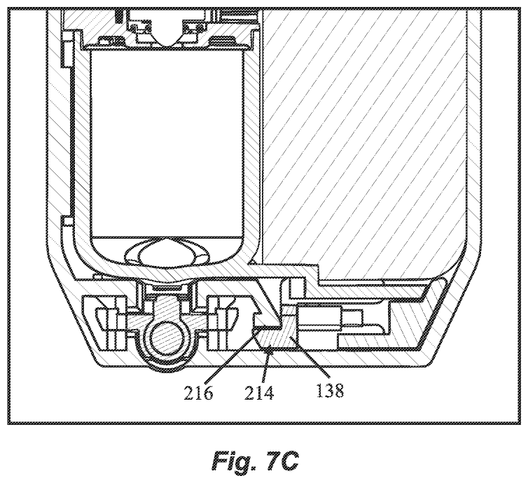

Although switch 106 on pump 100 includes raised projections 134 that can aid a user in manually sliding switch 106 between locked and unlocked positions, embodiments of pump 100 can be designed to automatically lock onto holder 200 when the pump is properly inserted onto the holder. A spring 136 can bias the switch into the locked position. FIG. 6A depicts the switch 106 in the locked position, FIG. 6B depicts the switch in the unlocked position (i.e., during insertion) and FIG. 6C depicts the switch in an automatic unlock position during removal, described in more detail below. A tray locking feature 138 can be attached to and therefore movable with switch 106. FIGS. 7A-7C are cutaway views of system depicting aspects of the locking features of the system with other elements removed for sake of clarity. As the pump 100 is slid along the tray, an angled ramp feature 139 of the tray lock 138 encounters the angled locking ramp 212 on the pump holder 200 adjacent the guide rail 208 noted previously. As can be seen most clearly in FIG. 7B, as the tray lock 138 slides along the locking ramp 212, the locking ramp 212 overcomes the bias provided by spring 136 to slide the tray lock 138 and the switch 106 into the unlocked position of FIG. 6B. When the tray lock 138 has traveled beyond the locking ramp 212, the spring 136 causes the switch 106 to snap back to its biased, locked position such that the tray lock 138 automatically nests into a locking aperture 214 in tray as shown in FIG. 7C. The pump 100 is then prevented from being inadvertently slid back off of the holder 200 by the interaction of tray lock 138 with the distal end 216 of locking ramp 212. In this manner, the pump 100 therefore automatically becomes releasably locked onto the pump holder 200 at the completion of the insertion process.

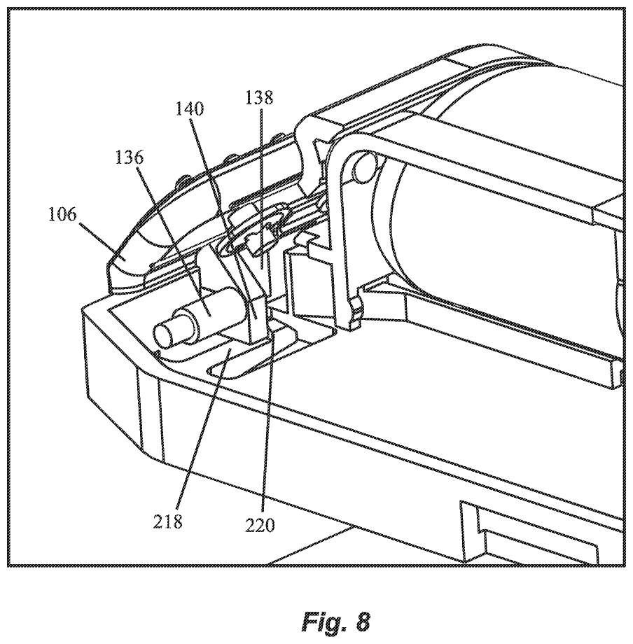

A user can manually unlock the pump 100 from the holder 200 by moving the switch to and holding the switch 106 in the unlocked position in order to remove the pump 100 from the holder 200 by sliding the pump 100 back off of the tray body 202. However, embodiments disclosed herein can also include an automatic unlock feature. One embodiment of such a feature is depicted in FIG. 8 (with certain elements removed for the sake of clarity). Rather than having to hold the switch 106 in the unlocked position to remove the pump, when the user slides the switch 106 to the unlocked position, a boss 140 on the tray lock 138 of the switch 106 is configured to catch and be held on a ledge 218 of holder 200 against a flange 220. This keeps the switch 106 in the unlocked position with the tray lock 138 of the switch 106 pulled out of the locking aperture 214 of the holder 200. The user can then slide the pump 100 back off of the tray body 202 without having to hold the switch 106 in the unlocked position against the bias of the spring 136, which enables one handed removal of pump 100. As the pump 100 is removed, the boss 140 will slide passed the flange 220 and the switch 106 will return to the default locked state is the pump continues to be removed. In some embodiments, if the user decides not to remove the pump after unlocking, provision of sufficient force on switch 106 can cause the boss 140 on the tray lock 138 to pass over the flange 220 to re-lock the pump.

In embodiments, a user-wearable infusion pump system can include a user-wearable infusion pump configured to contain a medicament and including a switch disposed on a perimeter of the user-wearable infusion pump. A pump holder can be configured to releasably hold the user-wearable infusion pump. The switch can automatically releasably lock the user-wearable infusion pump onto the pump holder when the user-wearable infusion pump is inserted onto the pump holder.

In some embodiments, the pump holder includes a tray having a perimeter shape generally matching a perimeter shape of the user-wearable infusion pump and an adhesive patch configured to releasably retain the pump holder on a body of a user.

In some embodiments, the tray includes one or more retaining walls extending upwardly around a perimeter of the tray.

In some embodiments, the tray includes an open end having no retaining wall to enable the user-wearable infusion pump to be slid horizontally onto the tray from the open end.

In some embodiments, the tray includes a guide rail extending upwardly from a base of the tray between two opposing retaining walls.

In some embodiments, the user-wearable infusion pump includes a lower guide rail on a bottom surface of the user-wearable infusion pump and the lower guide rail of the user-wearable infusion pump is configured to slide along the guide rail of the tray to guide the user-wearable infusion pump onto the tray.

In some embodiments, the switch is biased in a locked position.

In some embodiments, a guide feature on the pump holder causes the switch to slide to an unlocked position while the user-wearable infusion pump is being slid onto the pump holder.

In some embodiments, the guide feature is an angled ramp that interfaces with an angled surface connected to the switch to cause the switch to slide laterally from the locked position to the unlocked position.

In some embodiments, the switch automatically returns to the locked position when the user-wearable infusion pump is fully inserted onto the pump holder.

In some embodiments, the switch includes a tray lock that nests into a locking aperture on the pump holder when the switch returns to the locked position.

In some embodiments, the user-wearable infusion pump includes a drive unit and a cartridge selectively attachable to the drive unit, and the switch is disposed on the cartridge.

In some embodiments, the switch is configured to remain in the unlocked position if the switch is manually moved from the locked position to the unlocked position when the user-wearable infusion pump is fully inserted onto the pump holder.

In some embodiments, the switch remains in the unlocked position due to a tray lock on the switch being held against a flange on the pump holder preventing the switch from sliding back into the locked position while the user-wearable infusion pump is fully inserted onto the pump holder.

In embodiments, a user-wearable infusion pump configured to contain a medicament can include a switch disposed on a perimeter of the user-wearable infusion pump. The switch can be configured to automatically releasably lock the user-wearable infusion pump onto a pump holder when the user-wearable infusion pump is inserted onto the pump holder.

In some embodiments, the switch is biased in a locked position.

In some embodiments the user-wearable infusion pump includes a drive unit and a cartridge selectively attachable to the drive unit and the switch is disposed on the cartridge.

In embodiments, a pump holder configured to releasably hold a user-wearable infusion pump is configured to cause a switch on a user-wearable infusion pump to automatically releasably lock the user-wearable infusion pump onto the pump holder when the user-wearable infusion pump is inserted onto the pump holder.

In some embodiments, the pump holder is further configured to cause the switch to remain in an unlocked position if the switch is manually moved from the locked position to the unlocked position when the user-wearable infusion pump is fully inserted onto the pump holder.

In some embodiments, the pump holder includes a tray having a perimeter shape generally matching a perimeter shape of the user-wearable infusion pump and an adhesive patch configured to releasably retain the pump holder on a body of a user. The tray can include one or more retaining walls extending upwardly around a perimeter of the tray and an open end having no retaining wall to enable the user-wearable infusion pump to be slid horizontally onto the tray from the open end.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10,541,987; 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patent application Ser. Nos. 17/368,968; 17/896,492; 18/011,060; 18/071,814; 18/071,835; 18/075,029; 18/090,788 18/115,316; and Ser. No. 18/139,391.

For purposes of interpreting the claims, it is expressly intended that the provisions of 35 U.S.C. § 112(f) are not to be invoked unless the specific terms “means for” or “step for” are recited in a claim.

1. A user-wearable infusion pump system, comprising:
a user-wearable infusion pump configured to contain a medicament, the user wearable infusion pump including a switch disposed on a perimeter of the user-wearable infusion pump; and
a pump holder configured to releasably hold the user-wearable infusion pump,
wherein the switch automatically releasably locks the user-wearable infusion pump onto the pump holder when the user-wearable infusion pump is inserted onto the pump holder.

2. The user-wearable infusion pump system of claim 1, wherein the pump holder comprises a tray having a perimeter shape generally matching a perimeter shape of the user-wearable infusion pump and an adhesive patch configured to releasably retain the pump holder on a body of a user.

3. The user-wearable infusion pump system of claim 2, wherein the tray includes one or more retaining walls extending upwardly around a perimeter of the tray.

4. The user-wearable infusion pump system of claim 3, wherein the tray includes an open end having no retaining wall to enable the user-wearable infusion pump to be slid horizontally onto the tray from the open end.

5. The user-wearable infusion pump system of claim 2, wherein the tray includes a guide rail extending upwardly from a base of the tray between two opposing retaining walls.

6. The user-wearable infusion pump system of claim 5, wherein the user-wearable infusion pump includes a lower guide rail on a bottom surface of the user-wearable infusion pump, wherein the lower guide rail of the user-wearable infusion pump is configured to slide along the guide rail of the tray to guide the user-wearable infusion pump onto the tray.

7. The user-wearable infusion pump system of claim 2, wherein the switch is biased in a locked position.

8. The user-wearable infusion pump system of claim 7, where a guide feature on the pump holder causes the switch to slide to an unlocked position while the user-wearable infusion pump is being slid onto the pump holder.

9. The user-wearable infusion pump of claim 8, wherein the guide feature is an angled ramp that interfaces with an angled surface connected to the switch to cause the switch to slide laterally from the locked position to the unlocked position.

10. The user-wearable infusion pump system of claim 8, wherein the switch automatically returns to the locked position when the user-wearable infusion pump is fully inserted onto the pump holder.

11. The user-wearable infusion pump system of claim 10, wherein the switch includes a tray lock that nests into a locking aperture on the pump holder when the switch returns to the locked position.

12. The user-wearable infusion pump system of claim 1, wherein the user-wearable infusion pump includes a drive unit and a cartridge selectively attachable to the drive unit, and wherein the switch is disposed on the cartridge.

13. The user-wearable infusion pump system of claim 1, wherein the switch is configured to remain in the unlocked position if the switch is manually moved from the locked position to the unlocked position when the user-wearable infusion pump is fully inserted onto the pump holder.

14. The user-wearable infusion pump system of claim 13, wherein the switch remains in the unlocked position due to a tray lock on the switch being held against a flange on the pump holder preventing the switch from sliding back into the locked position while the user-wearable infusion pump is fully inserted onto the pump holder.

15. A user-wearable infusion pump configured to contain a medicament, the user wearable infusion pump including a switch disposed on a perimeter of the user-wearable infusion pump, wherein the switch is configured to automatically releasably lock the user-wearable infusion pump onto a pump holder when the user-wearable infusion pump is inserted onto the pump holder.

16. The user-wearable infusion pump of claim 15, wherein the switch is biased in a locked position.

17. The user-wearable infusion pump of claim 15, wherein the user-wearable infusion pump includes a drive unit and a cartridge selectively attachable to the drive unit, and wherein the switch is disposed on the cartridge.

18. A pump holder configured to releasably hold a user-wearable infusion pump, wherein the pump holder is configured to cause a switch on a user-wearable infusion pump to automatically releasably lock the user-wearable infusion pump onto the pump holder when the user-wearable infusion pump is inserted onto the pump holder.

19. The pump holder of claim 18, further configured to cause the switch to remain in an unlocked position if the switch is manually moved from the locked position to the unlocked position when the user-wearable infusion pump is fully inserted onto the pump holder.

20. The pump holder of claim 18, comprising a tray having a perimeter shape generally matching a perimeter shape of the user-wearable infusion pump and an adhesive patch configured to releasably retain the pump holder on a body of a user, wherein the tray includes one or more retaining walls extending upwardly around a perimeter of the tray and an open end having no retaining wall to enable the user-wearable infusion pump to be slid horizontally onto the tray from the open end.
